(19) United States
(12) Patent Application Publication
(10) Pub. No.: US 2022/0249497 A1
(43) Pub. Date: Aug. 11, 2022

(54) COMPOSITIONS AND METHODS FOR IMPROVING RELAXATION, SLEEP, COGNITION, AND/OR PHYSICAL PERFORMANCE

(71) Applicant: Health Via Modern Nutrition, Inc., San Francisco, CA (US)

(72) Inventors: Geoffrey Hubert Woo, San Francisco, CA (US); Latt Shahril Mansor, San Francisco, CA (US); Christine Ensley, San Francisco, CA (US)

(21) Appl. No.: 17/570,925

(22) Filed: Jan. 7, 2022

Related U.S. Application Data
(60) Provisional application No. 63/146,413, filed on Feb. 5, 2021.

(51) Int. Cl. A61K 31/522; A61K 31/047; A61K 31/198; A61P 25/28

(52) U.S. Cl. CPC A61K 31/522; A61P 25/28; A61K 31/198; A61K 31/047

(57) ABSTRACT

Cognitive and/or physical performance may be improved by administering a therapeutically effective amount of 1,3-butanediol, selected from R-1,3-butanediol, S-1,3-butanediol, and racemic 1,3-butanediol, in combination with a therapeutically effective amount of one or more additional active ingredients selected from caffeine, L-theanine, and a mixture of caffeine and L-theanine. Relaxation and/or sleep performance may be improved by administering a therapeutically effective amount of 1,3-butanediol, selected from R-1,3-butanediol, S-1,3-butanediol, and racemic 1,3-butanediol, in combination with a therapeutically effective amount of L-theanine. The disclosed compositions include synergistically effective amounts of 1,3-butanediol in combination with a synergistically effective amount of caffeine, L-theanine, and a mixture of caffeine and L-theanine. The compositions may be administered to a subject for improving relaxation, sleep, cognition, and/or physical performance in the subject.

### RELATED APPLICATIONS

This application claims priority to U.S. Provisional Patent Application Ser. No. 63/146,413, filed Feb. 5, 2021, which is incorporated herein in its entirety.

### FIELD OF THE INVENTION

The present invention relates to compositions and methods for improving relaxation, sleep, cognition, and/or physical performance. The invention relates to compositions comprising therapeutically effective amounts of 1,3-butanediol, present as R-1,3-butanediol, S-1,3-butanediol, or racemic 1,3-butanediol, and caffeine, compositions comprising therapeutically effective active amounts of 1,3-butanediol, present as R-1,3-butanediol, S-1,3-butanediol, or racemic 1,3-butanediol, and L-theanine, and compositions comprising therapeutically effective amounts of 1,3-butanediol, present as R-1,3-butanediol, S-1,3-butanediol, or racemic 1,3-butanediol, caffeine, and L-theanine. The compositions improve relaxation, sleep, cognition, and/or physical performance.

### BACKGROUND OF THE INVENTION

The present disclosure relates to compositions and methods for improving relaxation, sleep, cognition, and/or physical performance.

Relaxation refers to quieting and calming the mind and body. It includes releasing stress and tension in the body. Slower breathing and lower resting heartrate contribute to relaxation and sleep. Poor sleep or sleep deprivation can lead to anxiety and depression, impaired immune function, weight gain, and other undesirable health consequences. Improving relaxation and sleep contribute to improved health and wellness. It is desirable at all ages.

Cognition refers to a range of mental processes relating to the acquisition, storage, manipulation, and retrieval of information. It underpins many daily activities. Cognitive functions, refer to various mental abilities, including learning, thinking, reasoning, remembering, problem solving, decision making, and attention. Improving and preserving cognitive functions are desirable at all ages.

Physical activity is broadly defined as any bodily movement produced by skeletal muscles that requires energy expenditure. It encompasses all forms of activity, including walking and cycling, active play, work-related activity, active recreation, exercising, and competitive sports. Physical performance is typically measured by testing physical functions such as speed, strength, agility, coordination, etc. associated with physical activity. Improving and preserving physical performance is desirable at all ages.

Ketone bodies are the most energy-efficient fuel and yield more ATP per mole of substrate than pyruvate. Some recent studies have investigated ketones affecting cognitive and physical performance. Andrew J. Murray et al., “Novel ketone diet enhances physical and cognitive performance,” The FASEB Journal, Vol. 30, Issue 12, 2016, reported rats fed a diet supplemented with (R)-3-hydroxyburyl (R)-3-hydroxybutyrate as 30% of calories showed improved cognitive and physical performance. Mary T. Newport et al., “A new way to produce hyperketonemia: Use of ketone ester in case of Alzheimer's disease,” Alzheimer's & Dementia, Vol. 11, Issue 1, pp. 99-103, 2015, reported a 20-month oral administration of a ketone monoester to a patient with Alzheimer's disease dementia. The patient improved markedly in mood, affect, self-care, and cognitive and daily activity performance. The cognitive performance tracked plasma beta-hydroxybutyrate concentrations, with noticeable improvements in conversation and interaction at the higher levels, compared with predose levels. Mark Evans, et al., “Intermittent Running and Cognitive Performance after Ketone Ester Ingestion,” Medicine & Science in Sports & Exercise, November 2018, reported a 750 mg/kg ketone ester supplement attenuated the decline in executive function after exhausting exercise, suggesting a cognitive benefit after ketone ester ingestion. The ketone esters evaluated were R,S-1,3-butanediol acetoacetate ketone diester (KDE) and R-3-hydroxybutyl R-3-hydroxybutyrate ketone monoester (KME). It was reported that KDE would likely impair performance in high-intensity sports that demand a high rate of ATP provision from carbohydrate sources. Hunter S. Waldman et al., “Exogenous ketone salts do not improve cognitive responses after a high-intensity exercise protocol in healthy college-aged males,” Applied Physiology, Nutrition, and Metabolism, 16 Feb. 2018, examined the effects of a dl-β-hydroxybutyrate containing beverage on cognitive and performance measures during repeated Wingates (sprint interval training involving the repetition of “all out” 30 second efforts). No performance improvement was observed.

Studies have shown a relationship between caffeine consumption and cognitive performance (see Martin J. Jarvis, “Does caffeine intake enhance absolute levels of cognitive performance?” Psychopharmacology, 110, pp. 45-52, 1993). Cognitive performance in subjects was evaluated by measuring simple reaction time, choice reaction time, incidental verbal memory, and visuo-spatial reasoning (see H. J. Smit et al., “Effects of low doses of caffeine on cognitive performance, mood and thirst in low and higher caffeine consumers,” Psychopharmacology, 152, pp. 167-173, 2000). Cognitive performance in subjects was evaluated by measuring a long duration simple reaction time task and a rapid visual information processing task. Mood was evaluated in subjects by a mood questionnaire, which included an item on thirst (see Harris R. Lieberman et al., “Effects of caffeine, sleep loss, and stress on cognitive performance and mood during U.S. Navy SEAL training,” Psychopharmacology, 164, pp. 250-261, 2002). Cognitive performance in subjects was evaluated by measuring scanning visual vigilance, four-choice visual reaction time, a matching-to-sample working memory task, and a repeated acquisition test of motor learning and memory.

Some research indicates that L-theanine, a naturally occurring amino acid found in green tea, possesses relaxation benefits (see Suhyeon Kim et al., “GABA and L-theanine mixture decreases sleep latency and improves NREM sleep,” Pharm. Biol. 2019; 57(1): 65-73). Research indicates that L-theanine improves sleep in animals and humans (see Williams, J.; Kellett, J.; Roach, P. D.; McKune, A.; Mellor, D.; Thomas, J.; Naumovski, N. L-Theanine as a Functional Food Additive: Its Role in Disease Prevention and Health Promotion. Beverages 2016, 2, 13. https://doi.org/10.3390/beverages2020013). Research indicates a mixture of GABA and L-theanine has a positive synergistic effect on sleep quality and duration as compared to the GABA or 1-theanine alone (see Kim S, Jo K, Hong K B, Han S H, Suh H J. GABA and l-theanine mixture decreases sleep latency and improves NREM sleep. Pharm Biol. 2019;57(1):65-73. doi:10.1080/13880209.2018.1557698). Other research indicates L-theanine has a pronounced effect on attention performance and reaction time response in normal healthy subjects prone to have high anxiety (see Akiko Higashiyama, Hla Hla Htay, Makoto Ozeki, Lekh R. Juneja, Mahendra P. Kapoor, Effects of l-theanine on attention and reaction time response, Journal of Functional Foods, Volume 3, Issue 3,2011, Pages 171-778, ISSN 1756-4646, https://doi org/10.1016/j.jff.2011.03.009).

It would be an advancement in the art to provide novel and synergistic compositions and methods for improving relaxation, sleep, cognition, and/or physical performance. It would also be an advancement in the art to provide novel and synergistic compositions and methods for treating subjects in need of improved relaxation and/or experiencing a sleep, cognitive and/or physical performance disorder.

### BRIEF SUMMARY OF THE INVENTION

The present disclosure relates generally to compositions and methods for improving relaxation, sleep, cognition, and/or physical performance. The disclosed compositions include a therapeutically effective amount of 1,3-butanediol, in the form of R-1,3-butanediol, S-1,3-butanediol, or racemic 1,3-butanediol, with R-1,3-butanediol being presently preferred, in combination with a therapeutically effective amount of one or more additional active ingredients selected from caffeine, L-theanine, and a mixture of caffeine and L-theanine. The disclosed compositions include synergistically effective amounts of 1,3-butanediol, in the form of R-1,3-butanediol, S-1,3-butanediol, or racemic 1,3-butanediol, with R-1,3-butanediol being presently preferred, in combination with a synergistically effective amount of caffeine, L-theanine, or a mixture of caffeine and L-theanine. The disclosed compositions include a synergistic combination of compounds present in synergistically effective amounts. The disclosed compositions may be administered to a subject in a method of improving relaxation, sleep, cognition, and/or physical performance. The disclosed compositions may be administered to a subject in need of improved relaxation and/or for the treatment of a sleep, cognition, and/or physical disorder in the subject.

The disclosed compositions may include caffeine and/or L-theanine in isolated and purified chemical forms. The disclosed compositions may include caffeine and/or L-theanine in naturally occurring botanical forms. Non-limiting examples of naturally occurring botanical forms of caffeine include coffee beans, cacao (cocoa) beans, kola nuts, guarana berries, tea leaves, including green tea and black tea, yerba mate, and other caffeine-containing plant extracts. Non-limiting examples of naturally occurring botanical forms of L-theanine include tea leaves, including green tea and black tea, and other L-theanine-containing plant extracts.

As used herein, the expression [A], [B], [C], “and/or” [D] means that one or more of the cases connected by the expression “and/or” may occur individually or in combination. Thus, the expression means [A] or [B] or [C] or [D] may occur individually, or combinations of any two or more cases may occur, such as [A] and [B], [A] and [C], [B] and [C], [A], [C], and [D], etc.

1,3-butanediol has the formula HOCH₂CH₂CH(OH)CH₃. It is a chiral diol. As used herein, “1,3-butanediol” includes R-1,3-butanediol, S-1,3-butanediol, or racemic 1,3-butanediol. R-1,3-butanediol is presently preferred.

Various embodiments are described herein. It will be understood that the embodiments listed below may be combined not only as listed below, but in other suitable combinations in accordance with the scope of the invention.

One or more disclosed compositions for improving cognitive and/or physical performance include therapeutically effective amounts of R-1,3-butanediol, S-1,3-butanediol or racemic 1,3-butanediol and caffeine.

The therapeutically effective amount of R-1,3-butanediol, S-1,3-butanediol or racemic 1,3-butanediol (preferably R-1,3-butanediol) in the composition may range from about 1 mg to about 100 g. In one or more embodiments, the therapeutically effective amount of R-1,3-butanediol, S-1,3-butanediol or racemic 1,3-butanediol (preferably R-1,3-butanediol) may range from about 10 g to about 35 g.

The therapeutically effective amount of caffeine in the composition may range from about 1 mg to about 5 g. In one or more embodiments, the therapeutically effective amount of caffeine may range from about 100 mg to about 1 g. In one or more embodiments, the therapeutically effective amount of caffeine may range from about 200 mg to about 500 mg.

One or more disclosed combination compositions may have a ratio (w/w) of caffeine to R-1,3-butanediol, S-1,3-butanediol or racemic 1,3-butanediol (preferably R-1,3-butanediol) within a range from about 1:50 to about 1:350. A presently preferred ratio (w/w) of caffeine to R-1,3 -butanediol, S-1,3 -butanediol or racemic 1,3 -butanediol (preferably R-1,3 -butanediol) is about 1:100. For example, an efficacious ratio of caffeine to R-1,3-butanediol, S-1,3-butanediol or racemic 1,3-butanediol (preferably R-1,3-butanediol) may range from about 300 mg caffeine:15 g R-1,3-butanediol, S-1,3-butanediol or racemic 1,3-butanediol (preferably R-1,3-butanediol) to about 300 mg caffeine:60 g R-1,3-butanediol, S-1,3-butanediol or racemic 1,3-butanediol (preferably R-1,3-butanediol). A presently preferred ratio (w/w) of caffeine to R-1,3-butanediol, S-1,3-butanediol or racemic 1,3-butanediol (preferably R-1,3-butanediol) is about 100 mg caffeine:10 g R-1,3 -butanediol, S-1,3 -butanediol or racemic 1,3 -butanediol (preferably R-1,3 -butanediol).

One or more disclosed compositions for improving relaxation and sleep performance include therapeutically effective amounts of R-1,3-butanediol, S-1,3-butanediol or racemic 1,3-butanediol (preferably R-1,3-butanediol) and L-theanine. One or more disclosed compositions for improving cognitive and/or physical performance include therapeutically effective amounts of R-1,3 -butanediol, S-1,3 -butanediol or racemic 1,3 -butanediol (preferably R-1,3 -butanediol) and L-theanine.

The therapeutically effective amount of R-1,3-butanediol, S-1,3-butanediol or racemic 1,3-butanediol (preferably R-1,3-butanediol) in the composition may range from about 1 mg to about 100 g. In one or more embodiments, the therapeutically effective amount of R-1,3-butanediol, S-1,3-butanediol or racemic 1,3-butanediol (preferably R-1,3-butanediol) may range from about 10 g to about 35 g.

The therapeutically effective amount of L-theanine in the composition may range from about 1 mg to about 10 g. In one or more embodiments, the therapeutically effective amount of L-theanine may range from about 10 mg to about 1 g. In one or more embodiments, the therapeutically effective amount of L-theanine may range from about 100 mg to about 1 g or from about 200 mg to about 500 mg.

One or more disclosed compositions may have a ratio (w/w) of L-theanine to R-1,3-butanediol, S-1,3-butanediol or racemic 1,3-butanediol (preferably R-1,3-butanediol) within a range from about 1:50 to about 1:700. A presently preferred ratio (w/w) of L-theanine to R-1,3-butanediol, S-1,3-butanediol or racemic 1,3-butanediol (preferably R-1,3-butanediol) is about 1:100. For example, an efficacious ratio of L-theanine to R-1,3-butanediol, S-1,3-butanediol or racemic 1,3-butanediol (preferably R-1,3-butanediol) may range from about 300 mg L-theanine:15 g R-1,3 -butanediol, S-1,3 -butanediol or racemic 1,3 -butanediol (preferably R-1,3 -butanediol) to about 300 mg L-theanine:60 g R-1,3-butanediol, S-1,3-butanediol or racemic 1,3-butanediol (preferably R-1,3-butanediol) A presently preferred ratio (w/w) of L-theanine to R-1,3-butanediol, S-1,3-butanediol or racemic 1,3-butanediol (preferably R-1,3-butanediol) is about 100 mg L-theanine:10 g R-1,3-butanediol, S-1,3-butanediol or racemic 1,3-butanediol (preferably R-1,3-butanediol).

One or more disclosed compositions for improving cognitive and/or physical performance include therapeutically effective amounts of R-1,3-butanediol, S-1,3-butanediol or racemic 1,3-butanediol (preferably R-1,3-butanediol), caffeine, and L-theanine.

The therapeutically effective amount of R-1,3-butanediol, S-1,3-butanediol or racemic 1,3-butanediol (preferably R-1,3-butanediol) in the composition may range from about 1 mg to about 100 g. In one or more embodiments, the therapeutically effective amount of R-1,3-butanediol, S-1,3-butanediol or racemic 1,3-butanediol (preferably R-1,3-butanediol) may range from about 10 g to about 35 g.

The therapeutically effective amount of caffeine in the composition may range from about 1 mg to about 1 g. In one or more embodiments, the therapeutically effective amount of caffeine may range from about 10 mg to about 1 g. In one or more embodiments, the therapeutically effective amount of caffeine may range from about 100 mg to about 300 mg.

The therapeutically effective amount of L-theanine in the composition may range from about 1 mg to about 1 g. In one or more embodiments, the therapeutically effective amount of L-theanine may range from about 10 mg to about 1 g. In one or more embodiments, the therapeutically effective amount of L-theanine may range from about 100 mg to about 300 mg.

One or more disclosed compositions may have a ratio (w/w) of caffeine to R-1,3-butanediol, S-1,3-butanediol or racemic 1,3-butanediol (preferably R-1,3-butanediol) within a range from about 1:50 to about 1:350. A presently preferred ratio (w/w) of caffeine to R-1,3-butanediol, S-1,3-butanediol or racemic 1,3-butanediol (preferably R-1,3-butanediol) is about 1:100. For example, an efficacious ratio of caffeine to R-1,3-butanediol, S-1,3-butanediol or racemic 1,3-butanediol (preferably R-1,3-butanediol) may range from about 300 mg caffeine:15 g R-1,3-butanediol, S-1,3-butanediol or racemic 1,3-butanediol (preferably R-1,3-butanediol) to about 300 mg caffeine:60 g R-1,3-butanediol, S-1,3-butanediol or racemic 1,3-butanediol (preferably R-1,3-butanediol). A presently preferred ratio (w/w) of caffeine to R-1,3-butanediol, S-1,3-butanediol or racemic 1,3-butanediol (preferably R-1,3-butanediol) is about 100 mg caffeine: 10 g R-1,3 -butanediol, S-1,3 -butanediol or racemic 1,3 -butanediol (preferably R-1,3 -butanediol).

One or more disclosed compositions may have a ratio (w/w) of L-theanine to R-1,3-butanediol, S-1,3-butanediol or racemic 1,3-butanediol (preferably R-1,3-butanediol) within a range from about 1:50 to about 1:700. A presently preferred ratio (w/w) of L-theanine to R-1,3-butanediol, S-1,3-butanediol or racemic 1,3-butanediol (preferably R-1,3-butanediol) is about 1:100. For example, an efficacious ratio of L-theanine to R-1,3-butanediol, S-1,3-butanediol or racemic 1,3-butanediol (preferably R-1,3-butanediol) may range from about 300 mg L-theanine:15 g R-1,3-butanediol, S-1,3-butanediol or racemic 1,3-butanediol (preferably R-1,3-butanediol) to about 300 mg L-theanine:60 g R-1,3-butanediol, S-1,3-butanediol or racemic 1,3-butanediol (preferably R-1,3-butanediol). A presently preferred ratio (w/w) of L-theanine to R-1,3-butanediol, S-1,3-butanediol or racemic 1,3-butanediol (preferably R-1,3-butanediol) is about 100 mg L-theanine:10 g R-1,3-butanediol, S-1,3-butanediol or racemic 1,3-butanediol (preferably R-1,3-butanediol).

One or more disclosed compositions may have a ratio (w/w) of caffeine to L-theanine within a range from about 1:2 to about 2:1. A presently preferred ratio (w/w) of caffeine to L-theanine is about 1:1. For example, an efficacious ratio of caffeine to L-theanine may range from about 100 mg caffeine:200 mg L-theanine to about 100 mg caffeine:50 mg L-theanine. A presently preferred ratio (w/w) of caffeine to L-theanine is about 100 mg caffeine:100 mg L-theanine.

The disclosed invention includes one or more methods of improving cognitive and/or physical performance in a subject. The disclosed method includes administering to the subject any one of the disclosed compositions containing a therapeutically effective amount of 1,3-butanediol, in the form of R-1,3-butanediol, S-1,3-butanediol, or racemic 1,3-butanediol (preferably R-1,3-butanediol) and caffeine or a composition containing a therapeutically effective amount of 1,3-butanediol, in the form of R-1,3-butanediol, S-1,3-butanediol, or racemic 1,3-butanediol, (preferably R-1,3-butanediol), caffeine, and L-theanine or a composition containing a therapeutically effective amount of 1,3-butanediol, in the form of R-1,3-butanediol, S-1,3-butanediol, or racemic 1,3-butanediol, (preferably R-1,3-butanediol) and L-theanine.

The disclosed invention includes one or more methods of improving relaxation and sleep in a subject. The disclosed method includes administering to the subject any one of the disclosed compositions containing a therapeutically effective amount of 1,3-butanediol, in the form of R-1,3-butanediol, S-1,3-butanediol, or racemic 1,3-butanediol (preferably R-1,3-butanediol) and L-theanine.

The disclosed invention includes one or more methods of treating a cognitive or physical disorder in a subject. The disclosed method includes administering to the subject any one of the disclosed compositions containing a therapeutically effective amount of 1,3-butanediol, in the form of R-1,3-butanediol, S-1,3-butanediol, or racemic 1,3-butanediol (preferably R-1,3-butanediol) and caffeine, a composition containing a therapeutically effective amount of 1,3-butanediol, in the form of R-1,3-butanediol, S-1,3-butanediol, or racemic 1,3-butanediol (preferably R-1,3-butanediol), caffeine, and L-theanine, or a composition containing a therapeutically effective amount of 1,3-butanediol, in the form of R-1,3-butanediol, S-1,3-butanediol, or racemic 1,3-butanediol, (preferably R-1,3-butanediol) and L-theanine.

The disclosed invention includes one or more methods of treating a sleep disorder in a subject. The disclosed method includes administering to the subject any one of the disclosed compositions containing a therapeutically effective amount of 1,3-butanediol, in the form of R-1,3-butanediol, S-1,3-butanediol, or racemic 1,3-butanediol (preferably R-1,3-butanediol) and L-theanine.

In one or more of the disclosed methods, the composition is administered when relaxation is desired. In one or more of the disclosed methods, the composition is administered when sleep is desired. In one or more of the disclosed methods, the composition is administered when improved cognitive function in general is desired. In one or more of the disclosed methods, the composition is administered when improved physical performance in general is desired. In one or more of the disclosed methods, the composition is administered about 30 minutes before sleep is desired or commencing a cognitive and/or physically demanding task.

One or more of the disclosed compositions for improving cognitive and/or physical performance are synergistic compositions that include synergistically effective amounts of R-1,3-butanediol, S-1,3-butanediol or racemic 1,3-butanediol (preferably R-1,3-butanediol) and caffeine.

The synergistically effective amount of R-1,3-butanediol, S-1,3-butanediol or racemic 1,3-butanediol (preferably R-1,3-butanediol) in the composition may range from about 1 mg to about 100 g. In one or more embodiments, the synergistically effective amount of R-1,3-butanediol, S-1,3-butanediol or racemic 1,3-butanediol (preferably R-1,3-butanediol) may range from about 10 g to about 35 g.

The synergistically effective amount of caffeine in the composition may range from about 1 mg to about 5 g. In one or more embodiments, the synergistically effective amount of caffeine may range from about 100 mg to about 1 g. In one or more embodiments, the synergistically effective amount of caffeine may range from about 200 mg to about 500 mg.

One or more disclosed synergistic compositions may have a ratio (w/w) of caffeine to R-1,3-butanediol, S-1,3-butanediol or racemic 1,3-butanediol (preferably R-1,3-butanediol) within a range from about 1:50 to about 1:350. A presently preferred ratio (w/w) of caffeine to R-1,3-butanediol, S-1,3-butanediol or racemic 1,3-butanediol (preferably R-1,3-butanediol) is about 1:100. For example, an efficacious ratio of caffeine to R-1,3-butanediol, S-1,3-butanediol or racemic 1,3-butanediol (preferably R-1,3-butanediol) may range from about 300 mg caffeine:15 g R-1,3-butanediol to about 300 mg caffeine:60 g R-1,3-butanediol. A presently preferred ratio (w/w) of caffeine to R-1,3-butanediol, S-1,3-butanediol or racemic 1,3-butanediol (preferably R-1,3-butanediol) is about 100 mg caffeine:10 g R-1,3-butanediol, S-1,3-butanediol or racemic 1,3-butanediol (preferably R-1,3-butanediol).

One or more of the disclosed compositions for improving relaxation, sleep, cognition, and/or physical performance are synergistic compositions that include synergistically effective amounts of R-1,3-butanediol, S-1,3-butanediol or racemic 1,3-butanediol (preferably R-1,3-butanediol) and L-theanine.

The synergistically effective amount of R-1,3-butanediol, S-1,3-butanediol or racemic 1,3-butanediol (preferably R-1,3-butanediol) in the composition may range from about 1 mg to about 100 g. In one or more embodiments, the synergistically effective amount of R-1,3-butanediol, S-1,3-butanediol or racemic 1,3-butanediol (preferably R-1,3-butanediol) may range from about 10 g to about 35 g.

The synergistically effective amount of L-theanine in the composition may range from about 1 mg to about 10 g. In one or more embodiments, the synergistically effective amount of L-theanine may range from about 10 mg to about 1 g. In one or more embodiments, the synergistically effective amount of L-theanine may range from about 100 mg to about 1 g or from about 200 mg to about 500 mg.

One or more disclosed synergistic compositions may have a ratio (w/w) of L-theanine to R-1,3-butanediol, S-1,3-butanediol or racemic 1,3-butanediol (preferably R-1,3-butanediol) within a range from about 1:50 to about 1:700. A presently preferred ratio (w/w) of L-theanine to R-1,3-butanediol, S-1,3-butanediol or racemic 1,3-butanediol (preferably R-1,3-butanediol) is about 1:100. For example, an efficacious ratio of L-theanine to R-1,3-butanediol, S-1,3-butanediol or racemic 1,3-butanediol (preferably R-1,3-butanediol) may range from about 300 mg L-theanine: 15 g R-1,3-butanediol, S-1,3-butanediol or racemic 1,3-butanediol (preferably R-1,3-butanediol) to about 300 mg L-theanine:60 g R-1,3-butanediol, S-1,3-butanediol or racemic 1,3-butanediol (preferably R-1,3-butanediol). A presently preferred ratio (w/w) of L-theanine to R-1,3-butanediol, S-1,3-butanediol or racemic 1,3-butanediol (preferably R-1,3-butanediol) is about 100 mg L-theanine:10 g R-1,3-butanediol, S-1,3-butanediol or racemic 1,3-butanediol (preferably R-1,3-butanediol).

One or more of the disclosed compositions for improving cognitive and/or physical performance are synergistic compositions that include synergistically effective amounts of R-1,3-butanediol, S-1,3-butanediol or racemic 1,3-butanediol (preferably R-1,3-butanediol), caffeine, and L-theanine.

The synergistically effective amount of R-1,3-butanediol, S-1,3-butanediol or racemic 1,3-butanediol (preferably R-1,3-butanediol) in the composition may range from about 1 mg to about 100 g. In one or more embodiments, the synergistically effective amount of R-1,3-butanediol, S-1,3-butanediol or racemic 1,3-butanediol (preferably R-1,3-butanediol) may range from about 10 g to about 35 g.

The synergistically effective amount of caffeine in the composition may range from about 1 mg to about 1 g. In one or more embodiments, the synergistically effective amount of caffeine may range from about 10 mg to about 1 g. In one or more embodiments, the synergistically effective amount of caffeine may range from about 100 mg to about 300 mg.

The synergistically effective amount of L-theanine in the composition may range from about 1 mg to about 1 g. In one or more embodiments, the synergistically effective amount of L-theanine may range from about 10 mg to about 1 g. In one or more embodiments, the synergistically effective amount of L-theanine may range from about 100 mg to about 300 mg.

One or more disclosed synergistic compositions may have a ratio (w/w) of caffeine to R-1,3-butanediol, S-1,3-butanediol or racemic 1,3-butanediol (preferably R-1,3-butanediol) within a range from about 1:50 to about 1:350. A presently preferred ratio (w/w) of caffeine to R-1,3-butanediol, S-1,3-butanediol or racemic 1,3-butanediol (preferably R-1,3-butanediol) is about 1:100. For example, an efficacious ratio of caffeine to R-1,3-butanediol, S-1,3-butanediol or racemic 1,3-butanediol (preferably R-1,3-butanediol) may range from about 300 mg caffeine:15 g R-1,3-butanediol to about 300 mg caffeine:60 g R-1,3-butanediol, S-1,3-butanediol or racemic 1,3-butanediol (preferably R-1,3-butanediol). A presently preferred ratio (w/w) of caffeine to R-1,3-butanediol, S-1,3-butanediol or racemic 1,3-butanediol (preferably R-1,3-butanediol) is about 100 mg caffeine:10 g R-1,3-butanediol, S-1,3-butanediol or racemic 1,3-butanediol (preferably R-1,3 -butanediol).

One or more disclosed synergistic compositions may have a ratio (w/w) of L-theanine to R-1,3-butanediol, S-1,3-butanediol or racemic 1,3-butanediol (preferably R-1,3-butanediol) within a range from about 1:50 to about 1:700. A presently preferred ratio (w/w) of L-theanine to

R-1,3 -butanediol, S-1,3 -butanediol or racemic 1,3 -butanediol (preferably R-1,3 -butanediol) is about 1:100. For example, an efficacious ratio of L-theanine to R-1,3-butanediol, S-1,3-butanediol or racemic 1,3-butanediol (preferably R-1,3-butanediol) may range from about 300 mg L-theanine : 15 g R-1,3-butanediol, S-1,3-butanediol or racemic 1,3-butanediol (preferably R-1,3-butanediol) to about 300 mg L-theanine:60 g R-1,3-butanediol, S-1,3-butanediol or racemic 1,3-butanediol (preferably R-1,3-butanediol). A presently preferred ratio (w/w) of L-theanine to R-1,3-butanediol, S-1,3-butanediol or racemic 1,3-butanediol (preferably R-1,3-butanediol) is about 100 mg L-theanine:10 g R-1,3-butanediol, S-1,3-butanediol or racemic 1,3-butanediol (preferably R-1,3-butanediol).

One or more disclosed synergistic compositions may have a ratio (w/w) of caffeine to L-theanine within a range from about 1:2 to about 2:1. A presently preferred ratio (w/w) of caffeine to L-theanine is about 1:1. For example, an efficacious ratio of caffeine to L-theanine may range from about 100 mg caffeine:200 mg L-theanine to about 100 mg caffeine:50 mg L-theanine. A presently preferred ratio (w/w) of caffeine to L-theanine is about 100 mg caffeine:100 mg L-theanine.

The disclosed invention includes one or more methods of improving cognitive and/or physical performance in a subject. The disclosed method includes administering to the subject any one of the disclosed synergistic compositions containing a synergistically effective amount of 1,3-butanediol, in the form of R-1,3-butanediol, S-1,3-butanediol, or racemic 1,3-butanediol (preferably R-1,3-butanediol) and caffeine or a synergistic composition containing a synergistically effective amount of 1,3-butanediol, in the form of R-1,3-butanediol, S-1,3-butanediol, or racemic 1,3-butanediol (preferably R-1,3-butanediol), caffeine, and L-theanine or a synergistic composition containing a synergistically effective amount of 1,3-butanediol, in the form of R-1,3-butanediol, S-1,3-butanediol, or racemic 1,3-butanediol, (preferably R-1,3-butanediol) and L-theanine.

The disclosed invention includes one or more methods of improving relaxation and/or sleep in a subject. The disclosed method includes administering to the subject any one of the disclosed synergistic compositions containing a synergistically effective amount of 1,3-butanediol, in the form of R-1,3-butanediol, S-1,3-butanediol, or racemic 1,3-butanediol (preferably R-1,3-butanediol) and L-theanine.

The disclosed invention includes one or more methods of treating a cognitive and/or physical disorder in a subject. The disclosed method includes administering to the subject any one of the disclosed synergistic compositions a synergistically effective amount of 1,3-butanediol, in the form of R-1,3-butanediol, S-1,3-butanediol, or racemic 1,3-butanediol (preferably R-1,3-butanediol) and caffeine or a synergistic composition containing a synergistically effective amount of 1,3-butanediol, in the form of R-1,3-butanediol, S-1,3-butanediol, or racemic 1,3-butanediol (preferably R-1,3-butanediol), caffeine, and L-theanine or a synergistic composition containing a synergistically effective amount of 1,3-butanediol, in the form of R-1,3-butanediol, S-1,3-butanediol, or racemic 1,3-butanediol (preferably R-1,3-butanediol) and L-theanine.

The disclosed invention includes one or more methods of treating a sleep disorder in a subject. The disclosed method includes administering to the subject any one of the disclosed synergistic compositions containing a synergistically effective amount of 1,3-butanediol, in the form of R-1,3-butanediol, S-1,3-butanediol, or racemic 1,3-butanediol (preferably R-1,3-butanediol) and L-theanine.

In one or more of the disclosed methods, the synergistic composition is administered when relaxation is desired. In one or more of the disclosed methods, the synergistic composition is administered when sleep is desired. In one or more of the disclosed methods, the synergistic composition is administered when improved cognitive function in general is desired. In one or more of the disclosed methods, the synergistic composition is administered when improved physical performance in general is desired. In one or more of the disclosed methods, the synergistic composition is administered about 30 minutes before sleep is desired or commencing a cognitive and/or physically demanding task.

The disclosed compositions may be provided in any oral consumable form. It is within the scope of the disclosed invention to configure the disclosed compositions into formulations suitable for parenteral (including subcutaneous, intradermal, intramuscular, and intravenous) and rectal administration. In some embodiments, the disclosed compositions are in the form of a tablet, capsule, or pill suitable for oral administration. In some embodiments, the disclosed compositions are in liquid formulations (e.g., water, carbonated beverages, soft drinks, fermented beverages) suitable for oral administration. In some embodiments, the disclosed compositions are in the form of powders that can be used to prepare drink mixes or can be added as a supplement to other food or drink products. One or more pharmaceutically acceptable carriers may be provided. In various embodiments, the compositions are formulated for oral administration, including immediate release, extended release, and sustained release formulations.

It is to be understood that both the foregoing general description and the following detailed description are examples and explanatory and are not restrictive of the invention, as claimed. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE DRAWINGS

FIG. 1 is a bar graph showing the cognitive rating and physical rating of sixteen subjects for a composition of R-1,3-butanediol and caffeine.

FIG. 2 is a bar graph showing the cognitive rating and physical rating of five subjects for a composition of R-1,3-butanediol, caffeine, and L-theanine.

FIG. 3 is a set of bar graphs showing physical and mental energy/focus ratings of various subjects following consumption of Beverage 1 and Beverage 2.

### DETAILED DESCRIPTION OF THE INVENTION

The present invention relates to compositions and methods for improving relaxation and sleep. The present invention relates to compositions and methods for improving cognitive and/or physical performance. The invention relates to compositions comprising therapeutically effective amounts of R-1,3-butanediol, S-1,3-butanediol, or racemic 1,3-butanediol (preferably R-1,3-butanediol) and caffeine, compositions comprising therapeutically effective amounts of R-1,3-butanediol, S-1,3-butanediol, or racemic 1,3-butanediol (preferably R-1,3-butanediol) and L-theanine, and compositions comprising therapeutically effective amounts of R-1,3-butanediol, S-1,3-butanediol, or racemic 1,3-butanediol (preferably R-1,3-butanediol), caffeine, and L-theanine, which improve relaxation, sleep, cognition, and/or physical performance.

The terminology used herein is for the purpose of describing particular embodiments only and is not intended to be limiting. In this specification and in the claims that follow, reference will be made to a number of terms, which shall be defined to have the definitions set forth below. Unless otherwise defined, all technical and scientific terms used herein have the same meaning as commonly understood by one of ordinary skill in the art to which this invention belongs. The singular forms also include the plural unless the context clearly dictates otherwise. Thus, the singular forms “a,” “an” and “the” include plural referents unless the context clearly dictates otherwise.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment. Additionally, while the following description refers to several embodiments and examples of the various components and processes of the described invention, all of the described embodiments and examples are to be considered, in all respects, as illustrative only and not as being limiting in any manner.

Furthermore, the described features, structures, characteristics, processes, or methods of the invention may be combined in any suitable manner in one or more embodiments.

As used herein, unless explicitly stated otherwise or clearly implied otherwise, the term “about” refers to a range of values plus or minus 10 percent (“±10%”), e.g. about 1.0 encompasses values from 0.9 to 1.1.

“Active agent” and “therapeutic agent” means a compound that exerts a positive therapeutic effect on the health and well-being of a subject. Active agent can refer not only to a single active agent but also to a combination of two or more different active agents.

“Sustained release” and “extended release” means an active agent formulation that provides for gradual release of an active agent over an extended period of time, and typically, although not necessarily, results in substantially constant blood levels of an active agent over an extended time period.

“Dosage form” means any form of a composition for administration to a subject (typically a human seeking a therapeutic or synergistic effect). “Dose” refers to an amount of active agent. A single tablet or capsule is a unit dosage form. Multiple unit dosage forms can be administered to provide a therapeutically effective dose. A dosage form can include a combination of dosage forms.

“Effective amount” and “therapeutically effective amount” and “synergistically effective amount” refers to a nontoxic but sufficient amount of an active agent to achieve a desired therapeutic or synergistic effect.

The term “composition” refers to a composition that is suitable for administration to a subject. In general a “composition” is sterile, and preferably free of contaminants that are capable of eliciting an undesirable response within the subject.

The term “natural flavor” or “natural flavoring” means the essential oil, oleoresin, essence or extractive, protein hydrolysate, distillate, or any product of roasting, heating or enzymolysis, which contains the flavoring constituents derived from a spice, fruit or fruit juice, vegetable or vegetable juice, edible yeast, herb, bark, bud, root, leaf or similar plant material, meat, seafood, poultry, eggs, dairy products, or fermentation products thereof, whose significant function in food is flavoring rather than nutritional.

Percentages and ratios used herein, unless otherwise indicated, are by weight.

“Treating” and “treat” includes the administration to a subject one or more doses of an active agent to obtain a desired therapeutic or synergistic effect.

Certain compositions of the disclosed invention comprise a therapeutically effective amount of R-1,3 -butanediol, S-1,3 -butanediol, or racemic 1,3 -butanediol (preferably R-1,3 -butanediol) in combination with a therapeutically effective amount of one or more additional active ingredients selected from caffeine, L-theanine, and a mixture of caffeine and L-theanine. The compositions may also comprise one or more pharmaceutically acceptable (approved by a state or federal regulatory agency for use in humans, or is listed in the U.S. Pharmacopia, the European Pharmacopia) excipients or carriers. The terms “excipient” or “carrier” as used herein broadly refers to a biologically inactive substance used in combination with the active agents of the formulation. An excipient can be used, for example, as a solubilizing agent, a stabilizing agent, a diluent, an inert carrier, a preservative, a binder, a disintegrant, a coating agent, a flavoring agent, or a coloring agent. Preferably, at least one excipient is chosen to provide one or more beneficial physical properties to the formulation, such as increased stability and/or solubility to the therapeutic agents.

Non-limiting examples of suitable excipients for liquid or beverage formulations include flavoring agents, sweeteners, including nutritive and non-nutritive sweeteners, acidifiers such as citric, malic acid, tartaric acid, and phosphoric acid, and emulsifiers such as hydrocolloids like xanthan, gum acacia and gum acacia, modified starches, pectin, carrageenan, casein, and inulin. Non-limiting examples of suitable excipients for solid formulations include flow agents such as silicon dioxide, magnesium stearate, and stearic acid, binders such as guar gum, xanthan gum, and acacia gum, carriers such as naturally occurring complex carbohydrates, acidifiers such as naturally-occurring acids including citric acid, malic acid, tartaric acid, and aspartic acid.

Solutions and suspensions used for the delivery can include the following components: a sterile diluent such as water for injection, saline solution, fixed oils, polyethylene glycols, glycerin, polylene glycol, polysorbate, tocopherol polyethylene glycol succinate (TPGS), or other synthetic solvents; antibacterial agents such as benzyl alcohol or methyl parabens; antioxidants such as ascorbic acid or sodium bisulfite; chelating agents such as ethylenediamineteraacetic acid; buffers such as acetates, citrates or phosphates, and agents for the adjustment of tonicity such as sodium chloride or dextrose. The pH can be adjusted with acids or bases, such as hydrochloric acid or sodium hydroxide.

In some embodiments, the compositions for improving relaxation, sleep, cognition, and/or physical performance of the presently disclosed invention are prepared in a solid form such as a powder, tablet, pill or capsule for oral administration. In alternative embodiments, liquid formulations for oral administration may take such forms as water, carbonated beverages, soft drinks, fermented beverage suspensions, solutions and emulsions, and may contain formulatory agents such as suspending, stabilizing and/or dispersing agents. In some embodiments, the disclosed compositions may be in the form of powders that can be used to prepare drink mixes or can be added as a supplement to other food or drink products.

The dosage forms, e.g., an oral dosage form, may provide for rapid release or provide for extended release or sustained release, i.e., gradual, release of the R-1,3-butanediol, S-1,3-butanediol or racemic 1,3-butanediol (preferably R-1,3-butanediol) and the caffeine and/or L-theanine from the dosage form to the subject's body over an extended time period, typically providing for a substantially constant blood level of the two or more therapeutic agents over a time period in the range of about 4 to about 24 hours, typically in the range of about 4 to about 12 hours, or of about 6 to about 10 hours.

In some embodiments, it may be especially advantageous to formulate compositions of the invention in unit dosage form for ease of administration and uniformity of dosage. The term “unit dosage forms” as used herein refers to physically discrete units suited as unitary dosages for the individuals to be treated. That is, the compositions are formulated into discrete dosage units each containing a predetermined, “unit dosage” quantity of an active agent calculated to produce the desired therapeutic effect in association with the required pharmaceutical carrier. The specifications of unit dosage forms of the invention are dependent on the unique characteristics of the active agent to be delivered. Dosages can further be determined by reference to the usual dose and manner of administration of the ingredients. It should be noted that, in some cases, two or more individual dosage units in combination provide a therapeutically effective amount of the active agent, e.g., two tablets or capsules taken together may provide a therapeutically effective dosage of a first or second therapeutic agent, such that the unit dosage in each tablet or capsule is approximately 50% of the therapeutically effective amount.

Tablets may be manufactured using standard tablet processing procedures and equipment. Direct compression and granulation techniques are preferred. In addition to the active agent, tablets will generally contain inactive, pharmaceutically acceptable carrier materials such as binders, lubricants, disintegrants, fillers, stabilizers, surfactants, coloring agents, and the like.

Capsules are another oral dosage form of the present invention, wherein the R-1,3-butanediol, S-1,3-butanediol, or racemic 1,3-butanediol (preferably R-1,3-butanediol) and the caffeine and/or L-theanine are encapsulated in the form of a liquid or solid (including particulates such as granules, beads, powders or pellets). Suitable capsules may be either hard or soft, and are generally made of gelatin, starch, or a cellulosic material, with gelatin capsules preferred. Two-piece hard gelatin capsules are preferably sealed, such as with gelatin bands or the like.

Oral dosage forms, whether tablets, capsules, caplets, or particulates, if desired, may be formulated so as to provide for extended or controlled release of the R-1,3-butanediol, S-1,3-butanediol, or racemic 1,3-butanediol (preferably R-1,3-butanediol) and the caffeine and/or L-theanine.

Generally, as will be appreciated by those of ordinary skill in the art, extended release and sustained release dosage forms are formulated by dispersing at least one of the two or more active therapeutic agents within a matrix of a gradually hydrolyzable material such as a hydrophilic polymer, or by coating a solid, active agent-containing dosage form with such a material. Hydrophilic polymers useful for providing an extended release or a sustained release coating or matrix include, by way of example: cellulosic polymers such as hydroxypropyl cellulose, hydroxyethyl cellulose, hydroxypropyl methyl cellulose, methyl cellulose, ethyl cellulose, cellulose acetate, and carboxymethylcellulose sodium; acrylic acid polymers and copolymers, preferably formed from acrylic acid, methacrylic acid, acrylic acid alkyl esters, methacrylic acid alkyl esters, and the like, e.g. copolymers of acrylic acid, methacrylic acid, methyl acrylate, ethyl acrylate, methyl methacrylate and/or ethyl methacrylate; and vinyl polymers and copolymers such as polyvinyl pyrrolidone, polyvinyl acetate, and ethylene-vinyl acetate copolymer.

Other features and advantages of the present invention are apparent from the different examples that follow. The examples below illustrate different aspects and embodiments of the present invention and how to make and practice them. The examples do not limit the claimed invention. Although methods and materials similar or equivalent to those described herein can be used in the practice of the present invention, suitable methods and materials are described below. Based on the present disclosure the skilled artisan can identify and employ other components and methodology useful for practicing the present invention.

### EXAMPLES 1-6

### Therapeutically Effective Composition of R-1,3-Butanediol and Caffeine and/or L-theanine

Examples 1-6 describe a process for preparing several therapeutically effective compositions of R-1,3-butanediol and caffeine and/or L-theanine suitable for oral administration. The ingredients are mixed in water in the amounts set forth in Table 1. Citric acid (anhydrous) is used as the pH control. Thereafter, the composition is processed by heat until the liquid temperature reaches 165° F., bottled into a consumer-acceptable form, and cooled to a temperature of no more than 95° F.

TABLE 1

| Example | 1 | 2 | 3 | 4 | 5 | 6 |
|---|---|---|---|---|---|---|
| Water (mL) | 150 | 200 | 60 | 250 | 300 | 100 |
| R-1,3-Butanediol (g) | 10 | 15 | 5 | 10 | 12 | 15 |
| Caffeine (mg) | 100 | 200 | 100 | 150 | 0 | 200 |
| L-Theanine (mg) | 100 | 100 | 200 | 0 | 150 | 0 |
| Natural Flavor (mg) | 400 | 400 | 400 | 400 | 400 | 400 |
| pH Control (mg) | 60 | 65 | 35 | 70 | 70 | 50 |

### EXAMPLE 7

### Therapeutically Effective Composition of R-1,3-Butanediol and Caffeine, via Coffee Extract

This example describes a process for preparing a therapeutically effective composition of R-1,3-butanediol and caffeine suitable for oral administration. The composition is prepared by mixing 55 mL water and 10 g R-1,3-butanediol. 1.6 g soluble coffee, also known as instant coffee or coffee powder, containing therein 50 mg caffeine, is mixed with the composition until dissolved. 200 mg vanilla extract added to the composition. Thereafter, the composition is processed by heat until the liquid temperature reaches 165° F., bottled into a consumer-acceptable form, and cooled to a temperature of no more than 95° F.

### EXAMPLE 8

### Therapeutically Effective Composition of R-1,3-Butanediol and L-theanine, via Green Tea Extract, in Powder Mix Form

This example describes a process for preparing a therapeutically effective composition of R-1,3-butanediol and L-theanine in powder mix form, for oral administration after mixing with liquid by the consumer. The composition may be prepared by blending ingredients in powder form. Powder forms of individual ingredients or combinations of ingredients may be obtained by spray drying liquid forms of individual ingredients or combinations of ingredients.

In an example, the composition is prepared by spray drying a mixture of 15 g R-1,3-butanediol and 10 g gum acacia to form a powder. 500 mg of a powdered green tea extract, containing therein 50 mg L-theanine, is blended with the composition until evenly distributed. 500 mg of a powder FDA-compliant natural flavor and 1 g coconut cream powder are blended with the composition until evenly distributed. Thereafter, the composition is bottled into a consumer-acceptable form. In use, about 10 g of the resulting composition is mixed in 8 oz. water and administered to a subject.

### EXAMPLE 9

### Therapeutically Effective Composition of R-1,3-Butanediol and Caffeine

This example describes a process for preparing a therapeutically effective composition of R-1,3-butanediol and caffeine suitable for oral administration. The composition is prepared by mixing 1 g R-1,3-butanediol and 50 mg caffeine. Thereafter, the blend is encapsulated into gelatin softgel capsules and coated with lemon extract.

### EXAMPLE 10

### Therapeutically Effective Composition of R-1,3-Butanediol and Caffeine

This example describes a process for preparing beverage formulations comprising therapeutically effective amounts of R-1,3-butanediol and caffeine. A single serving (i.e., approximately 12 US fluid ounces) of a first beverage formulation was prepared by adding 5g R-1,3-butanediol and 150mg caffeine to water, a single serving (i.e., approximately 12 US fluid ounces) of a second beverage formulation was prepared by adding 10 g R-1,3-butanediol and 150mg caffeine water, and a single serving (i.e., approximately 12 US fluid ounces) of a third beverage formulation was prepared by adding 10 g R-1,3-butanediol and 200mg, wherein said first, second and third beverage formulations further comprised natural flavors, natural non-caloric sweeteners, and acidifiers. The first, second and third beverage formulations were carbonated and canned prior to administration. Mixing of the ingredients of the first, second and third beverage formulations can be accomplished by use of techniques and equipment that are known in the art.

### EXAMPLE 11

### Method for Improving Cognitive and/or Physical Performance

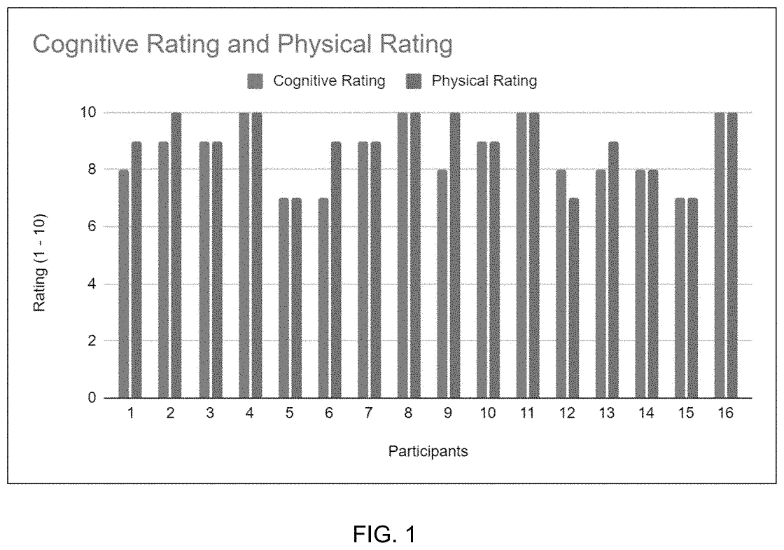

Sixteen subjects were administered a composition comprising 20-30 g R-1,3-butanediol and 100-200 mg caffeine. Approximately 30-60 minutes after consumption, the subjects were asked to go about their daily tasks. The subjects were asked to complete a questionnaire and rate their subjective feeling on cognitive and/or physical performance compared to other sources of caffeine such as coffee or energy drinks. The questionnaire asked the subjects to rate on a scale of 1-10, with 1 being “Less than normal” and 10 being “Higher than normal” (1) Levels of focus and mental energy (if applicable) and (2) Endurance level and physical energy (if applicable). Results of the study are reported in FIG. 1. The results report an average cognitive rating of 8.56 and an average physical rating of 8.94.

### EXAMPLE 12

### Method for Improving Cognitive and/or Physical Performance

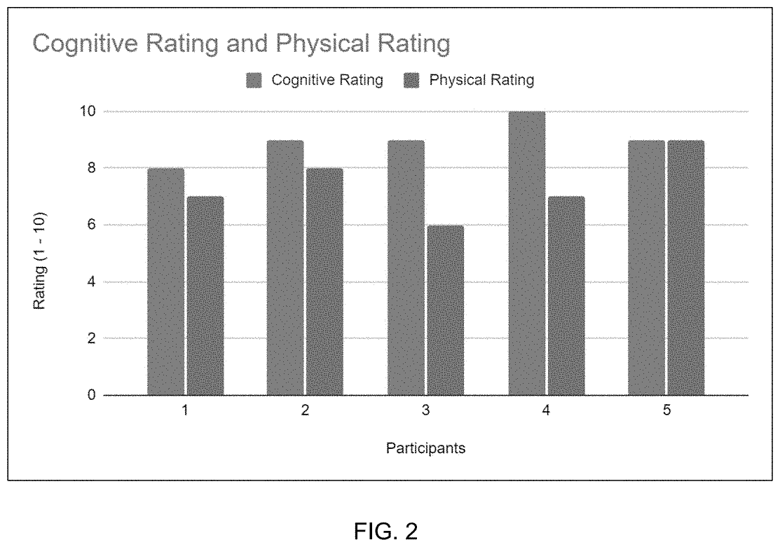

Five subjects were administered a composition comprising 20-30 g R-1,3-butanediol, 100-200 mg caffeine, and 100 mg L-theanine. Approximately 30-60 minutes after consumption, the subjects were asked to go about their daily tasks. The subjects were asked to complete a questionnaire and rate their subjective feeling on cognitive and/or physical performance compared to other sources of caffeine such as coffee or energy drinks. The questionnaire asked the subjects to rate on a scale of 1-10, with 1 being “Less than normal” and 10 being “Higher than normal” (1) Levels of focus and mental energy (if applicable) and (2) Endurance level and physical energy (if applicable). Results of the example are reported in FIG. 2. The results report an average cognitive rating of 9 and an average physical rating of 7.4.

### EXAMPLE 13

### Method for Improving Relaxation and/or Sleep Performance

Subjects are administered a composition comprising 20-30 g R-1,3-butanediol and 100 mg L-theanine. To evaluate relaxation performance, approximately 30-60 minutes after consumption, the subjects are asked to complete a questionnaire and rate their subjective feeling on relaxation. The questionnaire asks the subjects to rate on a scale of 1-10, with 1 being “Anxious” and 10 being “Very relaxed” the relaxation and anxiolytic effect (if applicable). To evaluate sleep performance, after awakening from sleep, the questionnaire asks the subjects to rate on a scale of 1-10, with 1 being “Poor” and 10 being “Very good” the quality of sleep performance (if applicable). The results show that the composition comprising R-1,3-butanediol and L-theanine improves relaxation and sleep performance.

### EXAMPLE 14

### Efficacy Study of R-1,3-Butanediol and Caffeine

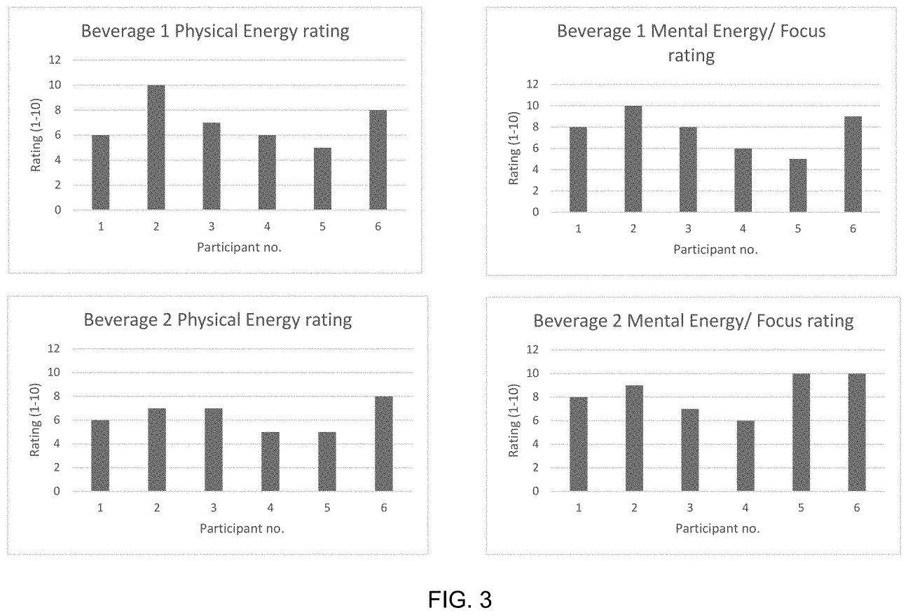

A blind study of the effects of the beverage formulations of Example 10 was carried out. Six subjects were administered the first beverage formulation (“Beverage 1”), six subjects were administered the second beverage formulation (“Beverage 2”), and 13 subjects were administered the third beverage formulation (“Beverage 3”). Approximately 30-60 minutes after consumption, the subjects were instructed to go about their daily tasks. Thirty minutes after consumption, the subjects were asked to complete a questionnaire and rate their subjective feelings, experience and/or effects resulting from Beverage 1 and Beverage 2. The questionnaire asked the subjects to rate on a scale of 1-10, with 1 being “Less than normal” and 10 being “Higher than normal” (1) Levels of focus and mental energy (if applicable) and (1) Endurance level and physical energy (if applicable). Results of the study are shown in FIG. 3. The results report an average cognitive rating of 8, and an average physical rating of 7 for Beverage 1, an average cognitive rating of 8 and an average physical rating of 6 for Beverage 2.

### EXAMPLE 15

### Testimonials of Efficacy of R-1,3-Butanediol and Caffeine

The following are testimonials from users of the beverage formulations of Example 10, according to the study of Example 14. Subjects were asked to “Please share any notes on the feeling/experience/effects of the drink”.
“I would say probably more focus as well. I'm someone that has ADHD and so I'm pretty in tune when I can feel like I'm focusing on flow versus not. And so, yeah, I would definitely say I've had significant changes since drinking it.” (J. D.—Beverage 2).
“So I did notice my energy level go up in kind of a spike, and I felt kind of good after drinking it, I'm not going to lie. I don't know exactly what I was feeling if there was a caffeine in that drink, but I felt like I kind of had a caffeine rush.” (R. V.—Beverage 2).
“My senses were more awake and I felt more alert.” (J. A.—Beverage 2).
“The first thing is I definitely felt I would probably say a little bit more relaxed, probably a little bit more sort of subdued; tranquil relaxation as opposed to, you know, normal fizzy drink that tend to have caffeine in it.” (J. P.—Beverage 2).
“I felt a very smooth, clean energy. Wasn't jittery but instead felt a boost to my focus and patience. I felt much more productive . . . I understand that this is because of the ketones, but it felt quite like magic. Like it unlocked a blocker I had in my brain. This feeling alone is worth me purchasing this again. I would recommend it to my friends as well.” (S. D.—Beverage 2).
“Super wired and dialed in. LOVE the effects. Shocked at how quickly I felt it. This is going to fly off the shelves.” (B. C.—Beverage 2).
“Around 15 minutes after completion I started to feel more focused and energized. The liftoff was smooth—not abrupt, which I like. Right now I am feeling more like tackling some work projects. So that is good. Likely this would also be good pre-workout . . . feeling good now.” (K. H.—Beverage 2).
“Went for a swim 30 minutes after consuming this beverage. Felt crisp, focused, and well fueled without feeling bloated or full. I'd call this a success.” (L. R.—Beverage 2).
“Definitely clearer thoughts and I specifically used it a little before I worked out, drank while working out, and then finished it at the end of the workout. The recovery has been great.” (W. O.—Beverage 2).
“Immediately after drinking I feel more clear headed without the usual effects of caffeine in causing anxiety.” (S. M.—Beverage 3).
“Feels great. Energized and mellow at the same time.” (M. B.—Beverage 3).
“I feel good . . . noticeable energy lift w/o feeling anxious or jittery.” (T. F.—Beverage 3).
“I definitely felt a mental buzz. I'm not usually cognitively aware of any change from caffeine/energy drinks; however, I was acutely aware of a change in how I felt within 20 minutes of drinking the product. I did feel more energetic afterwards which in turn pushed me to be more social.” (S. G.—Beverage 3).
“Feels very good.” (G. W.—Beverage 3).

### Synergistic Activity of R-1,3-Butanediol and Caffeine and/or L-theanine

Without being bound by theory, it is presently believed a synergistic effect is obtained by the co-administration of a synergistically effective amount of 1,3-butanediol, present as R-1,3-butanediol, S-1,3-butanediol, or racemic 1,3-butanediol (preferably R-1,3-butanediol), in combination with a synergistically effective amount of one or more additional active ingredients selected from caffeine, L-theanine, and a mixture of caffeine and L-theanine.

It is presently believed synergistic effects between R-1,3-butanediol, S-1,3-butanediol, or racemic 1,3-butanediol (preferably R-1,3-butanediol) and caffeine are obtained in cognitive improvement and physical performance improvement.

Cognitive Improvement

Caffeine works through the antagonism of adenosine receptors, especially in the central nervous system, and increased release of dopamine, noradrenalin, and glutamate. Caffeine is also found to increase brain activity and energy metabolism (see Da Chang et al., “Caffeine Caused a Widespread Increase of Resting Brain Entropy,” Sci Rep 8, 2700 (2018)).

Ketone is a unique substrate in that it is derived from fats, but unlike fats it can cross the blood brain barrier and enter the brain to provide energy directly to the brain. This has been shown to counter many neurological disorders due to mitochondrial dysfunction or glucose scarcity in the brain to that leads to the lack of substrate and energy in the brain and consequently inflammation, apoptosis and other adverse events that cause the disorders.

It is believed that when R-1,3-butanediol, S-1,3-butanediol, or racemic 1,3-butanediol (preferably R-1,3-butanediol) and caffeine are used together, the increased brain energy metabolism and activity stimulated by caffeine can be complemented by ketones as substrates to supply the energy the brain needs to function optimally.

Physical Performance Improvement

Caffeine can induce calcium release from the sarcoplasmic reticulum and can also inhibit its reuptake. This could affect neuromuscular function modulation and lead to contractile force increase in the skeletal muscles.

Exogenous ketones have previously showed improved physical performance (see Poffé C, Ramaekers M. Bogaerts S, Hespel P. Bicarbonate Unlocks the Ergogenic Action of Ketone Monoester Intake in Endurance Exercise. Med Sci Sports Exerc. 2020 Jul. 27; Cox P J, Kirk T, Ashmore T. Willerton K, Evans R, Smith A, Murray A J, Stubbs B, West J, McLure S W, King M T Dodd MS, Holloway C, Neubauer S, Drawer S, Veech R L, Griffin J L, Clarke K. Nutritional Ketosis Alters Fuel Preference and Thereby Endurance Performance in Athletes. Cell Metab. 2016 Aug. 9;24(2):256-68). The exact mechanism has not been determined, but the proposed mechanisms include glycogen sparing effect, improved recovery, and decrease in fatigue perception.

As caffeine increases contractile force in skeletal muscles, ketones may be synergistic in at least two aspects. First ketone is a fast and clean source of energy for muscle contractions and secondly, ingesting ketones may be able to sustain the ergogenic effects longer via sparing of glycogen stores.

It is presently believed synergistic effects between R-1,3-butanediol, S-1,3-butanediol, or racemic 1,3-butanediol (preferably R-1,3-butanediol) and L-theanine are obtained in at least relaxation, sleep, and/or cognitive improvement.

Relaxation and Sleep

Some research indicates that L-theanine possesses relaxation benefits (see Suhyeon Kim et al., “GABA and L-theanine mixture decreases sleep latency and improves NREM sleep,” Pharm. Biol. 2019; 57(1): 65-73). Research indicates that L-theanine improves sleep in animals and humans (see Williams, J.; Kellett, J.; Roach, P. D.; McKune, A.; Mellor, D.; Thomas, J.; Naumovski, N. L-Theanine as a Functional Food Additive: Its Role in Disease Prevention and Health Promotion. Beverages 2016, 2, 13. https://doi.org/10.3390/beverages2020013). Research suggests a mixture of GABA and L-theanine has a positive synergistic effect on sleep quality and duration as compared to the GABA or L-theanine alone (see Kim S, Jo K, Hong K B, Han S H Sub H J. GABA and l-theanine mixture decreases sleep latency and improves NREM sleep. Pharm Biol. 2019;57(1):65-73. doi:10.1080/13880209.2018.1557698).

Cognitive Improvement

Animal neurochemistry studies suggest that L-theanine increases brain serotonin, dopamine, GABA levels. In addition, behavioral studies in animals suggest improvement in learning and memory. L-theanine has antipsychotic-like and possibly antidepressant-like effects (see Nathan P J, Lu K, Gray M, Oliver C. The neuropharmacology of L-theanine(N-ethyl-L-glutamine): a possible neuroprotective and cognitive enhancing agent. J Herb Pharmacother. 2006;6(2):21-30). It exerts these effects, at least in part, through induction of brain derived neurotrophic factor (BDNF) in the hippocampus. BDNF, a trophic factor associated with cognitive improvement and the alleviation of depression and anxiety (see Wakabayashi C, Numakawa T, Ninomiya M, Chiba S, Kunugi H. Behavioral and molecular evidence for psychotropic effects in L-theanine, Psychopharmacology (Berl). 2012 February;219(4):1099-109). Other research indicates L-theanine has a pronounced effect on attention performance and reaction time response in normal healthy subjects prone to have high anxiety (see Akiko Higashiyama, Hla Hla Htay, Makoto Ozeki, Lekh R. Juneja, Mahendra P. Kapoor, Effects of l-theanine on attention and reaction time response. Journal of Functional Foods, Volume 3, Issue 3, 2011, Pages 171-178, ISSN 1756-4646, https://doi.org/10.1016/j.jff.2011.03.009).

Ketone bodies have also been found to increase brain synaptosomal GABA content (see Erecińska M, Nelson D, Daikhin Y, Yudkoff M. Regulation of GABA level in rat brain synaptosomes: fluxes through enzymes of the GABA shunt and effects of glutamate, calcium, and ketone bodies. J Neurochem. 1996 December;67(6):2325-34). On top of that, Ketones have also been proven to promote expression of BDNF (see Sama F. Sleiman et al., Exercise promotes the expression of brain derived neurotrophic factor (BDNF) through the activation of the ketone body β-hydroxybutyrate,” eLife 2016;5:315092, Sang Woo Kim et al., Ketone beta-hydroxybutyrate up-regulates BDNF expression through NF-κB as an adaptive response against ROS, which may improve neuronal bioenergetics and enhance neuroprotection, Neurology April 2017, 88 (16 Supplement) P3.090).

Preliminary data suggest that L-theanine and ketones work synergistically to provide anxiolytic effects (via the BDNF pathway) in addition to the improvement in learning and memory. This can also be enhanced with the stimulation of caffeine on increased brain activity and energetics.

All publications, patents, patent applications, or other documents cited herein are hereby incorporated by reference in their entirety for all purposes to the same extent as if each individual publication, patent, patent application, or other document was individually indicated to be incorporated by reference for all purposes.

While this disclosure has been particularly shown and described with references to preferred embodiments thereof, it will be understood by those skilled in the art that various changes in form and details may be made therein without departing from the scope of the disclosure encompassed by the appended claims.

What is claimed is:

1. A composition comprising:
1,3-butanediol selected from R-1,3-butanediol, S-1,3-butanediol, and racemic 1,3-butanediol in an amount from 1 mg to 100 g; and
caffeine in an amount from 10 mg to 1 g.

2. The composition according to claim 1, comprising from 5 g to 35 g 1,3-butanediol and from 50 mg to 300 mg caffeine.

3. The composition according to claims 1, comprising a ratio (w/w) of caffeine to 1,3-butanediol within a range from 1:50 to 1:350.

4. The composition according to claim 1, further comprising L-theanine in an amount from 10 mg to 1 g.

5. The composition according to claim 4, comprising from 100 mg to 300 mg L-theanine.

6. The composition according to claim 4, comprising a ratio (w/w) of caffeine to L-theanine within a range from 1:2 to 2:1.

7. The composition according to claim 1, wherein the 1,3-butanediol is R-1,3-butanediol.

8. The composition according to claim 1, wherein said composition is prepared in a beverage form.

9. A method of improving cognitive and/or physical performance in a subject, comprising:
administering to the subject the composition according to claim 1.

10. The method according to claim 8, wherein the composition comprises about 10 g R-1,3-butanediol and about 200 mg caffeine and the composition is administered about 30 minutes before a cognitively and/or physically demanding task, not more than three times per day.

11. A synergistic composition comprising synergistically effective amounts of 1,3-butanediol, selected from R-1,3-butanediol, S-1,3-butanediol, and racemic 1,3-butanediol, and caffeine.

12. The synergistic composition according to claim 11, comprising a ratio (w/w) of caffeine to 1,3-butanediol within a range from 1:50 to 1:700.

13. The synergistic composition according to claim 110, comprising from 1 mg to 100 g 1,3-butanediol and from 10 mg to 1 g caffeine.

14. The synergistic composition according to claim 13, comprising from 10 g to 35 g 1,3-butanediol and from 100 mg to 300 mg caffeine.

15. The synergistic composition according to claim 11, wherein the 1,3-butanediol is R-1,3-butanediol.

16. The synergistic composition according to claim 11, further comprising a synergistically effective amount of L-theanine.

17. The synergistic composition of claim 16, comprising from 50 mg to 400 mg L-theanine.

18. The synergistic composition according to claim 16, comprising a ratio (w/w) of caffeine to L-theanine within a range from 1:2 to 2:1.

19. The synergistic composition according to claim 11, wherein said synergistic composition is prepared in a beverage form.

20. A synergistic product comprising a synergistic amount of 1,3-butanediol, selected from R-1,3-butanediol, S-1,3-butanediol, and racemic 1,3-butanediol, and caffeine as a combined preparation for simultaneous use in the treatment of a cognitive and/or physical disorder in a subject.

21. The synergistic product according to claim 20, wherein the synergistic amount comprises from 1 mg to 100 g 1,3-butanediol and from 10 mg to 1 g caffeine.

22. The synergistic product according to claim 20, wherein said synergistic product is prepared in a beverage form.

23. The synergistic product according to claim 21, wherein the synergistic amount comprises from 5 g to 35 g 1,3-butanediol and from 50 mg to 300 mg caffeine.

24. The synergistic product according to claim 20, comprising a ratio (w/w) of caffeine to 1,3-butanediol within a range from 1:50 to 1:350.

25. The synergistic product according to claim 20, further comprising L-theanine in an amount from 10 mg to 1 g.

26. The synergistic product according to claim 25, comprising from 100 mg to 300 mg L-theanine.

27. The synergistic product according to claim 25, comprising a ratio (w/w) of caffeine to L-theanine within a range from 1:2 to 2:1.

28. The synergistic product according to claim 20, wherein the 1,3-butanediol is R-1,3-butanediol.

29. A method of treating a cognitive or physical disorder in a subject, comprising administering to the subject a synergistic product comprising about 10 g R-1,3-butanediol and about 200 mg caffeine, wherein the synergistic product is administered about 30 minutes before a cognitively and/or physically demanding task, not more than three times per day.
